(19) United States
(12) Patent Application Publication
(10) Pub. No.: US 2015/0159133 A1
(43) Pub. Date: Jun. 11, 2015

(54) METHOD OF IN VITRO DIFFERENTIATION OF MOTOR NEURON PROGENITORS (MNPS) FROM HUMAN INDUCED PLURIPOTENT STEM CELLS AND CRYOPRESERVATION OF MNPS

(71) Applicants: Fan Yang, Frederick, MD (US); Huan Tran, Boyds, MD (US); Thomas Fellner, Gaithersburg, MD (US)

(72) Inventors: Fan Yang, Frederick, MD (US); Huan Tran, Boyds, MD (US); Thomas Fellner, Gaithersburg, MD (US)

(73) Assignee: LONZA WALKERSVILLE, INC., Walkersville, MD (US)

(21) Appl. No.: 14/406,665

(22) PCT Filed: Jun. 11, 2013
(86) PCT No.: PCT/US13/45184

§ 371 Date: Dec. 9, 2014

Related U.S. Application Data
(60) Provisional application No. 61/658,061, filed on Jun. 11, 2012.

(51) Int. Cl. C12N 5/0793

(52) U.S. Cl. CPC C12N 5/0619

(57) ABSTRACT

Methods are disclosed for the initiation and differentiation of human embryonic stem cells (hESCs) and induced pluripotent stem cells (iPSCs) into motor neuron progenitor cells (MNPs). Methods are also disclosed for the cryopreservation of MNPs. The methods particularly relate to the simple, efficient, scalable, and reproducible generation, and subsequent frozen maintenance, of MNPs for downstream therapeutic applications. The methods can be used for the production of MNPs from various lines of hESCs and iPSCs.

### CROSS-REFERENCE TO RELATED APPLICATIONS

This application claims priority to U.S. Provisional Application Ser. No. 61/658,061 titled “METHOD OF IN VITRO DIFFERENTIATION OF MOTOR NEURON PROGENITORS (MNPS) FROM HUMAN INDUCED PLURIPOTENT STEM CELLS AND CRYOPRESERVATION OF MNPS,” filed on Jun. 11, 2012, which is incorporated herein, in its entirety, by this reference.

### FIELD OF THE INVENTION

The present invention relates to producing motor neuron progenitors (MNPs) from human induced pluripotent stem cell (iPSC) lines and human embryonic stem cell (hESC) lines. More particularly, this invention provides a method of producing greater than 75-90% purity and functional MNPs from various hESC lines and iPSC lines. The present invention also relates to cryopreserving MNPs. More particularly, this invention provides a method of cryopreserving MNPs that allows more than 90% recovery of highly viable and functional cells post-thawing.

### BACKGROUND OF THE INVENTION

Neurons may be classified based on their structure and function. Structural classification is based on the number of processes extending from the neuronal cell body. In contrast, functional classification is based on the direction in which the neuron transmits nerve impulses. Motor neurons (MN) are efferent neurons that convey nerve impulses from the brain and spinal cord (that is, away from the central nervous system) to effectors (which may be either muscles or glands). The satellite-shaped cell body of the MN is connected to a single, long axon (which forms a neuromuscular junction with effectors) and several shorter dendrites projecting out of the cell body. Neurology and drug discovery laboratories have traditionally relied on rodent models for the study of MN function and associated diseases such as amyotrophic lateral sclerosis (also known as ALS or Lou Gehrig's disease) and spinal muscular atrophy. However, recent advances in stem cell research, such as the availability of hESCs and iPSCs, offer the opportunity to develop human models for the study of MNs and associated diseases.

Current MN differentiation protocols for hESCs and iPSCs rely on embryoid body formation and directed differentiation by stromal feeder cell co-culture or by selective survival conditions. Current methods for generating MNs (Hu et al. 2010; Karumbayaram et al. 2009, Bouniting et al. 2011; US 2011/0091927A1) Nature Biotechnology 29:279-286 2011 are similar to each other and, in general, all include generating embryoid bodies or aggregates from either hESC or iPSCs in the presence of retinoic acid and induction of the sonic hedgehog pathway. The aggregates are then maintained in culture with neurotrophic factors to promote MN survival. Okano and Shimazaki (U.S. Pat. No. 7,294,510) have described another method of differentiating of ES cell to neural stem cell using noggin to form embryoid bodies. Subsequently, the embryoid bodies are subjected to suspension culture in the presence of fibroblast growth factor and a sonic hedgehog protein without using retinoic acid to induce formation of neural stem cells. Finally, neural stem cells are differentiating into only motor neurons and GABAeric neurons without glia cells contamination. Other directed differentiation methods without embryoid body/aggregate formation (Karumbayaram et al. 2009) Stem Cells; 27(4) 806-811; 2009 showed successful generation MN at very low efficiency.

Current methods are complicated, lengthy, and result in low yields. Zhang and Li (U.S. Pat. No. 7,588,937) have also described a method of producing spinal motor neuron by using hESCs growing on mouse embryonic fibroblast feeders as starting point. These cells are formed embryoid bodies in suspension and continue to differentiate in rosettes structure using retinoic acid and retinoic acid together with sonic hedgehog. The yield MNPs from this method was in ˜20-50% purity. U.S. Pat. No. 8,137,971 discloses the most efficient method available to date to make MNs from hESCs. However, this method cultures hESC in feeder cell-free conditions. It is difficult to consistently achieve sufficient neural induction to efficiently produce MNPs. Furthermore, this method only works with one hESC line and produces MNPs at approximately 65% purity. Therefore, this process is not as efficient as indicated. Other studies (Hu et al. 2010; Karumbayaram et al. 2009, Bouniting et al. 2011; PNAS 107(9) 4335-4340; 2010; Stem Cells; 27(4) 806-811; 2009; Nature Biotechnology 29:279-286 2011 have successfully demonstrated that MNPs can be generated from a number of iPSC lines; however, these studies are highly variable and inefficient. Currently, there is no optimized process to reproducibly generate MNPs from iPSCs that is simple, efficient, and scalable. Therefore, there is a need for a simple and efficient method of producing MNPs from various lines of hESCs and iPSCs. In addition, there is a need for a method of producing MNPs that is scalable and reproducible for downstream therapeutic applications.

In addition, there is no method available for the cryopreservation of MNPs with efficient recovery. The current method of cryopreservation for MNPs uses high concentration of DMSO with a serum free basal medium supplemented with B27 and freezing is performed by using a freezing container such as Nalgene® “Mr. Frosty” (available through Sigma-Aldrich) in the presence of isopropanol and mechanical −80° C. freezer which provides a slow cooling rate of about −1° C./min to −80° C. and subsequent plunging in liquid nitrogen. Although, this simple freezing method would work for cryopreserving of MNPs and other cell types, the recovery of MNPs after thawing is not always consistent and never reaches >90% cell viability. This could be due to the fact that the freezing container has no control for the cooling rate. This cooling rate is depending on the mechanical −80 C freezer's ability to keep its temperature. Furthermore, it also depends on the location within the freezer since there is temperature variability within a freezer shelf and shelf locations. Therefore, there is also a need for a method of cryopreserving MNPs that allows the frozen cells to be thawed with consistent high viability and functionality.

### SUMMARY OF THE INVENTION

Accordingly, an object of the present invention is to provide a method of producing MNPs from human pluripotent cells (including hESCs and iPSCs) that is simple, efficient, scalable, and reproducible.

A further object of this invention is to provide a method of cryopreserving MNPs that allows the frozen cells to be thawed with high viability and functionality.

A further object of this invention is to provide methods applicable to pluripotent stem cells in general, and induced pluripotent stem cells in particular.

A further object of this invention is to provide universal methods applicable regardless of cell source.

A further object of this invention is to provide for higher robustness and viability of MNPs.

A further object of this invention is to provide for greater than 90% viability of recovered MNPs.

A further object of this invention is to reduce the clumping of the cells.

A further object of this invention is to provide a method of cryopreserving MNPs that allows the frozen cells to be thawed with high recovery.

A further object of this invention is provide a method including the use of mouse embryonic fibroblast (MEF) feeder cells to culture the iPSCs and hESCs

In one representative embodiment, a method is provided for producing MNPs in vitro by harvesting hESCs or iPSC, which have been grown on mouse embryonic fibroblasts for at least 6-7 days, and these undifferentiated hESCs or iPSCs are neuralized by plating and culturing in ultra-low-adherence flasks containing a serum-free motor neuron induction medium for about 5 days. The induction medium is a classical medium containing growth factor, non-essential amino acids, L-glutamine, insulin, transferrin, selenium and B27 and is supplemented with bFGF and retinoic acid to generate spheres. The cultured spheres are ventralized by using an induction medium supplemented with low concentration of bFGF for about 10 days to promote formation of neurospheres. The suspension cultured neurospheres are mechanically dissociated into smaller spheres and expanded on an adherent surface for about 5 days as neural rosettes or early stage MNP. Thereafter, the adherent early stage MNPs are dissociated using trypsin solution and are transferred to gelatin-coated flasks containing induction medium to further enrich MNP cells. The non-adherent cells are collected from the gelatin-coated flasks, re-plated as adherent cells in matrix-coated flasks, such as Matrigel®, containing induction medium, and repeatedly cultured and re-fed with induction medium for about 5-6 days. Then, the adherent late stage MNPs are harvested from the matrix-coated flasks using trypsin solution. The resulting cell suspension is transferred to gelatin-coated flasks to further enrich MNP cells and remove contaminant cells. Non-adherent MNPs from the gelatin-coated flasks are collected, and large cell clumps are sedimented in a conical tube. MNPs are collected from the supernatant in the conical tube following the sedimentation of the large cell clumps can be stored as cryopreserved MNPs.

In an aspect of one representative embodiment, the concentration of bFGF is used at 10 ng/ml in the induction medium for the first day of plating of undifferentiated hESC or iPSC through day 7 of culture, and bFGF concentration is decreased to 5 ng/ml on day 8 of culture until harvesting of the MNPs.

In an aspect of one representative embodiment, the concentration of retinoic acid is used at 10 μM from day 1 through day 7 culture.

In another representative embodiment, the harvested MNPs-containing supernatant is centrifuged and the resulting cell pellet of MNPs is re-suspended in a cold protein-free and serum-free freezing medium pre-formulated with DMSO in a cryovial. An example of such freezing media can include CRYOSTORE® CS10 solution, by BioLife of Seattle, Wash. By way of example, 10% DMSO can also be used and optimized to freeze cells. The cryovial is transferred to a controlled rate freezer and subjected to programmed freezing process. Thereafter, the cryovial is transferred from the controlled rate freezer to a liquid nitrogen Dewar for long term storage.

Typically, upon reconstitution of cryopreserved stem, cells no more than 70% of the cells are viable post-thaw. It is a further aspect of the cryopreservation of the MNPs as disclosed herein that, upon reconstitution, 70% or greater and specifically 90% or greater of the cells are viable post thaw.

These and other objects are achieved in the present invention. The present invention overcomes a major disadvantage of current methods of producing MNPs for therapeutic applications by providing a simple, highly efficient, scalable, and reproducible method of differentiating MNPs from various lines of hESCs and iPSCs. One invention disclosed herein is the use of mouse embryonic fibroblast (MEF) feeder cells to culture the iPSCs and hESCs. hESCs were originally derived and cultured on MEF layers which permit continuous growth of hESCs in an undifferentiated stage (Amit et al 2003; Biology of Reprod 68:2150-2156). In vitro, the hESCs tended to differentiate when cultured in the absence of MEF feeder layers (Thomson et al 1998 Science 282 (5391) 1145-7). Feeder cells have also been derived from several human cell types such as human foreskin fibroblasts or adult Fallopian type epithelial cells (Amit et al 2003 Biology of Reproduct 22(5) 1231-8; Richard et al 2002 Nat Biotech 20(9) 933-6; Richards et al 2003. Stem Cells 21(5)546-56; Hovatta et al 2003. Human Reproduction 18(7) 1404-9; Choo et al 2004. Biotech and Bioengineering 88(3) 321-33). However, the ability of different types of human feeder cells to support the undifferentiated growth of hESCs varies (Richards et al 2003 Stem Cells 21(5)546-56; Eiselleova et al 2008. J of Devel Biol 52(4) 353-63). Activin A and basic fibroblast growth factor (bFGF) are key factors in maintenance the pluripotent state of stem cells (Eiselleova et al 2008. J of Devel Biol 52(4) 353-6315; Xiao et al 2006. Stem Cells 24(6) 1476-86). Mouse feeder cells express more Activin A than human feeder cells, but they do not express bFGF like human feeder cells (Eiselleova et al 2008. J of Devel Biol 52(4) 353-6315). When compared to human feeder cells, MEFs seem to support better the undifferentiated growth of some hESC lines, whereas more spontaneous differentiation and a lower proportion of SSEA3 positive cells can be observed with human feeder cells (Eiselleova et al 2008. J of Devel Biol 52(4) 353-6315). Cultured feeder cells secrets numerous of uncharacterized growth factors, cytokines, extracellular matrix (ECM) components such as proteoglycans, fibronectin, various types of collagen, nidogen, and laminin. These aforementioned ECM proteins, growth factors and cytokines secreted by feeder cells provide hESC/iPSCs a scaffold for hESC/iPSCs to anchor and provide the signals to proliferate and maintain their pluripotency. The present inventors have unexpectedly found that using feeder cells maintains the pluripotent state of the iPSCs and hESCs such that these cells are primed in better conditions for subsequent neuralization and ventralization steps of motor neuron differentiation process. This important change in methodology over the currently available art improves neuralization potency and results in a functional and more homogenous population of MNPs. In addition, the method of the present invention can be used for a variety of iPSC and hESC lines with consistent yield of high purity MNPs. Accordingly, the method of the present invention has significant improvements over the current technology that requires culturing of hESCs under feeder cell-free conditions such as matrix gel (U.S. Pat. No. 8,137,971).

The present invention also introduces a highly efficient freezing method for MNPs that includes the use of chemically-defined cryoprotectants and a controlled rate freezer to improve cell recovery after thawing from long term storage. The latter improvement over the prior art allows the long term storage of MNPs, which provides greater experimental flexibility in downstream applications. Cryopreservation during the differentiation process introduces efficiencies for the commercial manufacture of MNPs.

There has thus been outlined, rather broadly, features of the invention in order that the detailed description thereof that follows may be better understood, and in order that the present contribution to the art may be better appreciated. There are, of course, additional features of the invention that will be described further hereinafter. Indeed, it is to be understood that both the foregoing general description and the following detailed description are exemplary and explanatory and are intended to provide further explanation of the invention as claimed.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other methods, systems, kits, and compositions for carrying out the several purposes of the present invention. It is important, therefore, that equivalent constructions insofar as they do not depart from the spirit and scope of the present invention, are included in the present invention.

The accompanying drawings are included to provide a further understanding of the invention and are incorporated in and constitute a part of this specification, illustrate several embodiments of the invention and together with the description serve to explain the principles of the invention.

### BRIEF DESCRIPTION OF THE DRAWINGS

FIG. 1 illustrates an MNP manufacturing process.

FIGS. 2A-E illustrates: (A) cultured iPSCs on MEFs feeder; (B) neurospheres in suspension culture condition after caudalization on day 8; (C) neurospheres in suspension culture after ventralization on day 18; (D) Plated neurospheres on adherent substrate showing migration of the early MN progenitors; and (E) expansion of the early MN progenitors after first purification.

FIGS. 3A-C provide images of characterization of MNPs before cryopreservation on day 28: (A) MNP specific marker Islet1; (B) MNP specific marker HB9; and (C) neurofilament protein (Tuj1).

FIGS. 4A-C illustrate images of MN progenitors after cryopreservation and re-plated on PDL/laminin coated surface on day 3 after thawing: (A) Thawed MN progenitors after cryopreservation with branched morphology; (B) neurofilaments (Tuj1, green) and GFAP (red) and DAPI nuclear staining (blue); and (C) HB9 (red) transcription factor makers for MN progenitor and neurofilament (Tuj1, green).

FIG. 5 illustrates maturation of MNP in the absence and presence of B27 in laminin coating step.

### DETAILED DESCRIPTION OF THE INVENTION

Provided herein are methods for the production of MNPs from various lines of hESCs and iPSCs.

Reference will now be made in detail to representative embodiments of the invention, examples of which are illustrated in the accompanying drawings.

### Listing of Materials and Equipment

The following is a list of materials, reagents, and equipment used in the present invention. One skilled in the art understands that the materials, reagents, and equipment of the present invention are provided as examples only and that similarly functioning materials, reagents, and equipment can be substituted for those in the list without undue experimentation. Equipment list: laminar flow biosafety cabinet class II, centrifuge, water bath, incubator, refrigerator, first freezer, second freezer, pipet aid or pipet ball, multi-channel pipettors, multi-channel aspirator, microscope, assorted pipettors and pipet tips, hemocytometer, NucleoCounter® (Lonza), Cell scrappers (Corning 3010), T-25 flasks (Corning 430639), T-75 flasks (Corning 430641), T-75 ultra-low adherence flasks (Corning 3814), 15 ml centrifuge tubes (BD Falcon 352097), 50 ml centrifuge tubes (BD Falcon 352098), aspirating pipets (BD Falcon 357558), assorted serological pipets (BD Falcon 356543, 357551, 357525, 357550), 50 ml Steriflip® tubes (Millipore SCGP00525), 50 ml reagent reservoirs, precision balance, assorted Nalgene® bottles (Fisher Scientific 339072,2923145, 0292500A, and 0292500B), cryovials (Corning 430659), Technicloth® Wipes, Trypan blue solution, 70% Isopropanol, 2% Bacdown® (Fisher Scientific 04-355-13), water for cell culture (Lonza), hESC medium, MNP basal medium, MNP Plating medium, MNP maintenance medium, 10 μg/ml bFGF stock solution (BioSource PHG0263 (1 mg powder)), Retinoic acid (Sigma-Aldrich R2625), TrypLE® solution (Gibco 12605-010) Recombinant porcine tryp sin formulated in DPBS and 1 mM EDTA, PBS: Phosphate buffer saline, low osmolality medium without w/o L-glutamine or Hepes buffer for hESCs and iPSCs, Poly-D-lysine (Sigma-Aldrich P7405-5MG), Laminin solution (Roche 11243217001), DMEM High Glucose (Thermo Fisher SH3008101), Collagenase type IV lyophilized (Gibco 17104-019), an optimized, cGMP produced, protein-free and serum-free freezing medium pre-formulated with DMSO, laminin working solution, Poly-D-Lysine stock solution, Poly-D-lysine working solution, collagenase IV working solution and retinoic acid stock solution.

In at least one embodiment, the water bath is about 37° C.

In at least one embodiment the incubator is capable of maintaining 37° C.±2° C. with a 5%±2% CO₂ and humidified atmosphere.

In at least one embodiment the refrigerator is capable of maintaining about 2 to 8° C.

In at least one embodiment the first freezer is capable of maintaining about −20 to −30° C.

In at least one embodiment the second freezer is capable of maintaining about −78 to −82° C.

In at least one embodiment, the hESC medium is Knockout DMEM supplemented with 20% KOSR, glutamax, non-essential amino acids and bFGF and beta mercaptoethanol, MNP Induction medium such as a 50:50 mixture of classical DMEM high glucose and DMEM/F12 supplemented with insulin, transferrin, selenium, glutamine, magnesium chloride and B27 (NSF1). Once mixed, the medium was used for no more than 14 days.

In at least one embodiment, the MNP basal medium is a classical DMEM high glucose supplemented with B27 (NSF1), non-essential amino acids, insulin, transferrin, selenium, hepes, magnesium chloride, zinc sulfate, and copper sulfate with or without L-glutamine. Once mixed, the medium was used for no more than 14 days.

In at least one embodiment, the MNP Plating medium is MNP basal medium supplemented with B27 (NSF1) and L-glutamine.

In at least one embodiment, the MNP maintenance medium is MNP basal medium supplemented with B27 (NSF1).

In at least one embodiment, low osmolity medium is KnockOut DMEM/F12 (Gibco 12660-012).

In at least one embodiment, the an optimized, cGMP produced, protein-free and serum-free freezing medium pre-formulated with DMSO is CryoStor® CS10 (BioLife® Solution 210102).

In at least one embodiment, the laminin working solution is stock laminin solution (500 μg/ml) diluted by mixing 300 μl of stock laminin solution in 10 ml of MNP basal medium (15 μg/ml).

In at least one embodiment, ten sterile cryovials were labeled and stored at −20° C., 30 minutes prior to use. Five mg of Poly-D-Lysine powder from Sigma was rehydrated in 10 ml of water for cell culture for at least 30 minutes in the laminar flow hood. The rehydrated poly-D-lysine stock solution (500 μg/ml) was aliquoted at 1 ml/vial and the aliquots were stored at −20° C. until required for coating.

In at least one embodiment, a 1 ml aliquot of the stock Poly-D-Lysine solution was thawed and diluted by mixing it in 9 ml of PBS. The working solution of poly-D-lysine (50 μg/ml) was used for coating.

In at least one embodiment, collagenase IV working solution is 0.1 ml/cm2 of 1 mg/mL collagenase solution (7.5 ml per T-75 flask and 2.5 ml per T-25 flask). An appropriate amount of milligrams of collagenase powder was weighed out using the precision balance by multiplying by 2 the calculated volume of collagenase solution. The weighed out collagenase was transferred into a 50 ml tube and the calculated volume of low osmolarity DMEM/F12 medium, e.g. Knockout™ DMEM/F12 from Life Technologies, was added. The solution was mixed thoroughly by swirling until all the collagenase was completely dissolved, and was filtered through a 0.22 μm filter before use. The solution was stored at 4° C. and used within one week.

In at least one embodiment, care was taken while preparing retinoic acid aliquots to avoid exposing the materials to light for too long. In one embodiment, aliquots were prepared in the laminar flow hood as quickly as possible with the hood lights turned off. The 100 mg vial of retinoic acid was disinfected and placed it in the laminar flow hood. The top of the glass vial was broken off carefully and discarded into a sharps bin. Using a 1 ml syringe filter, 1 ml of dimethylsulfoxide (DMSO) solution was added to dissolve the retinoic acid powder. The mixture was transferred into a 50 ml tube and the glass vial was rinsed three times with 1 ml DMSO each rinse. The rinses were transferred to the 50 ml tube containing the retinoic acid mixture. Another 12.6 ml of DMSO were added to the tube and mixed well by pipetting up and down several times. This made a stock solution of 20 mM retinoic acid. Using a 200 μl pipette, 100 μl aliquots of stock solution were made in 500 μl amber tubes and the tubes were placed immediately in a −80° C. freezer.

### Example 1

### Differentiation of hESCs

Materials and Experimental Design

Initiation of Motorneuron Differentiation Day 0

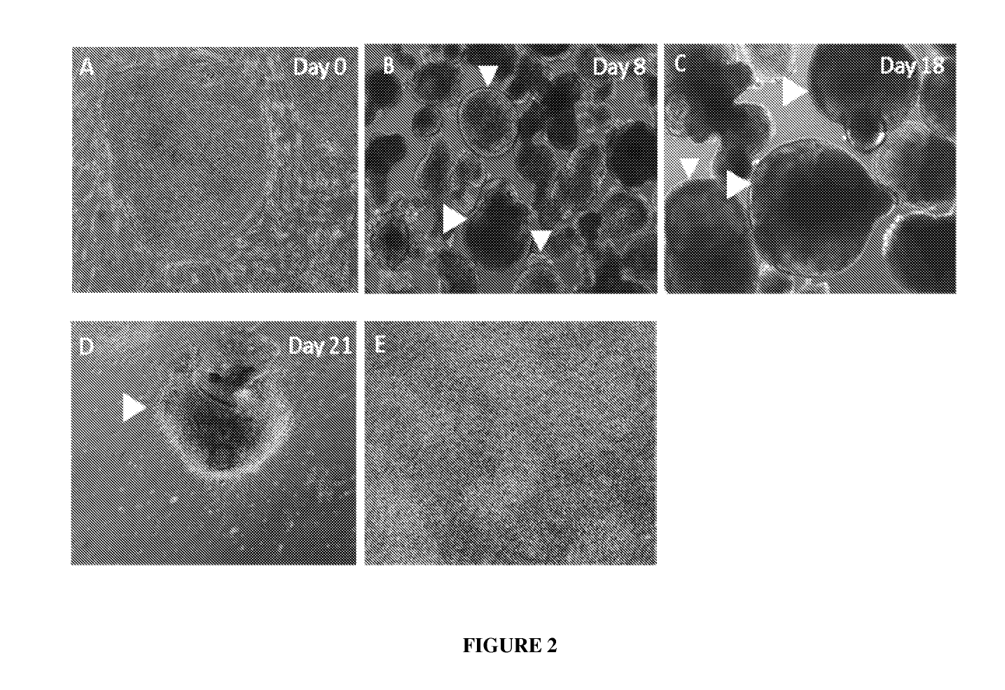

Human ESCs or iPSCs were co-cultured with MEF feeder cells with hESC growth medium (knockout DMEM/F12, 20% KSR, Glutamax, NEAA, BME and bFGF) on the T75 flask (FIG. 2A). hESCs were initiated when the cell density reached around 80% confluence. Spent medium from the T-75 flask was replaced with 30 ml of a 1:1 mixture of hESC medium and MNP induction medium supplemented with 10 ng/ml bFGF. After 24 hours, the hESCs/iPSCs colonies were dissociated using a collagenase solution (1 mg/ml) and the dissociated cells were suspended with MNP induction medium supplemented with 10 ng/ml bFGF and 10 μM of retinoic acid. The cell suspension then was transferred into an ultra-low-adherence T-75 flask.

Medium was gently replaced daily for 7 days without breaking cellular aggregates (FIG. 2B). Cell suspension along with the spent medium was transferred into a 50 ml conical tube. The cellular aggregates (spheres) were allowed to settle and the spent medium and cell debris were aspirated carefully without losing any spheres.

Starting from day 8, retinoic acid was removed from medium and bFGF concentration was reduced to 5 ng/mL. Numerous neurospheres were formed while some have the tendency to attach to other cells and form larger cellular spheres (FIG. 2C). Medium was replaced every other day until day 20 by using the same procedure but at a shorter sediment time to remove non-neurospheres.

On Day 20, the spheres along with the spent medium were transferred into a 50 ml conical tube. The spheres were allowed to sediment for about 30 seconds and the spent medium was aspirated. The spheres were resuspended in 5-7 ml fresh medium and they were sedimented for about 15-30 seconds while spent medium was aspirated. This washing step was repeated twice. The spheres were pipetted gently with a 10 ml serological pipet to break up the aggregates and were transferred to Matrigel®-coated T-75 flasks and distributed evenly prior to placing in the incubator.

Neurospheres were adhered to Matrigel®-coated surface, and the cells migration from spheres and cells with unipolar or bipolar extension could be observed (FIG. 2D). Medium was replaced every other day.

After 5 days, the cultures were dissociated with TrypLE solution. Once the cells were dissociated, to each T-75 flask, 15 ml of MNP induction medium supplemented with bFGF was added and the cell aggregates were triturated gently to break up the remaining spheres. The cell suspension was centrifuged for 3 minutes at 200×g. The cells were resuspended with 10 ml of fresh MNP induction medium supplemented with bFGF and the suspension was transferred into the gelatin-coated T-75 flask. The T-75 flask was incubated for 15 minutes at 37° C. undisturbed. After incubation, all the non-adherent cells were collected from the T-75 flask and transferred to another gelatin-coated T-75 flask. The T-75 flask was incubated for 15 minutes at 37° C. undisturbed. All the non-adherent cells were collected from the T-75 flask and the cell suspension was distributed evenly in the two Matrigel®-coated T-75 flasks (about 20 ml/T-75 flask) and one 4-well chamber slide pre-coated with Matrigel®. The seeded flasks and chamber were placed in the incubator.

Medium was replaced every other day. On Day 28, the slides were fixed by 4% paraformaldehyde and stained with neuronal marker Tuj1 and specific motor neuron makers including: Hb9, Islet1.

On Day 30, MNPs (FIG. 2E) were harvested by using the same methods as described in previous section of Day 25 MNP using trypLE and purified by using gelatin coated flasks to remove non MNP cells. MNPs were collected as non-adherent cells from gelatin coated flasks. Cell counts and viability were determined by using NucleoCounter.

Results

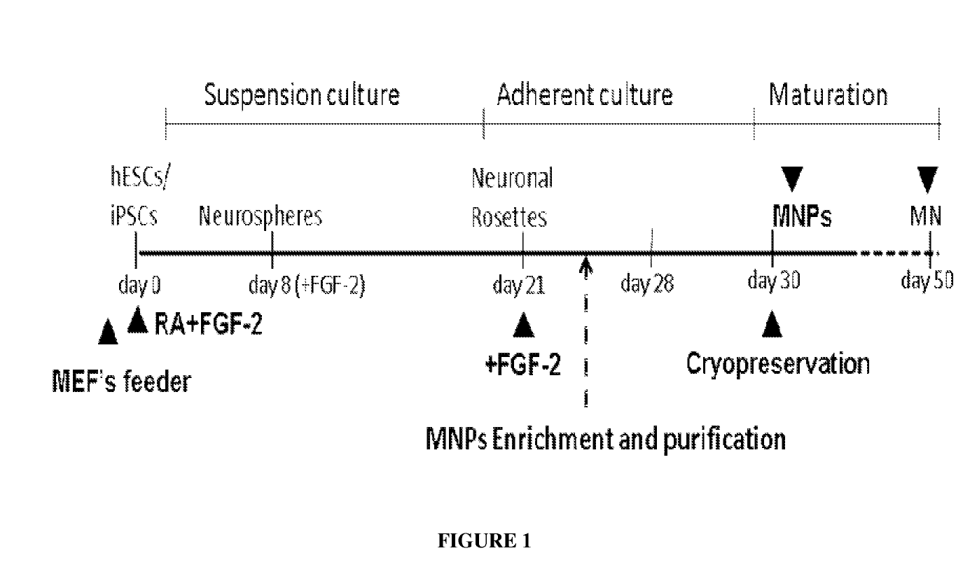

Generation of Motor neurons in vertebrate animal involves: neuralization of ectodermal cells, caudalization of the neuroectodermal cells and ventralization of the caudalized neural progenitors. FIG. 1 provides an overview of the MNP differentiation process. To initiate neuralization, a chemically defined formulation is introduced: 50:50 mixture of classical DMEM high glucose and DMEM/F12 supplemented with insulin, transferrin, selenium, glutamine, magnesium chloride and B27 (NSF1). Then the physical environment of the hES cells/iPSCs changed from adherent feeder cells to a non-adherent aggregates condition (suspension), the cell suspension was cultured for next 20 days in low adherent containers.

Caudalization and ventralization were induced by using retinoic acid (RA). Cellular aggregates are treated with RA from day 1 for 7 days. Following RA treatment, bFGF concentration was reduced to 5 ng/ml and frequency of medium changes was extended from daily feeding to 2-days feeding schedule. This could facilitate accumulation of autocrine factors from neural progenitors. Not to be bound by theory, it is believed the autocrine factors are important for motor neuron differentiation.

After neurospheres formation, the suspension was plated onto Matrigel®-coated surface for further expansion. During this time, the elongated cells with radial arrangements migrated from the spherical formations (rosettes) along with some flat cells would outgrow as well. Early MNPs could be purified by using negative adsorption on gelatin coated surface where contaminating cells adhered and MNPs were separated as non-adherent cells.

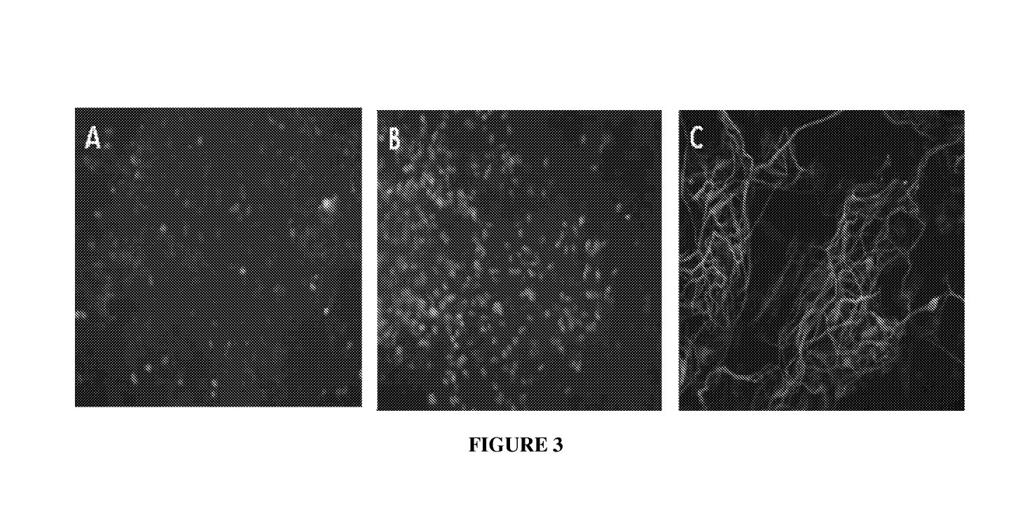

After expansion and purification, MNPs are characterized with neuronal marker and MNPs specific markers before cryopreservation. The results showed that on day 28, majority cells expressed HB9, Islet1 and Tuj1 (FIG. 3). PSCs marker such as Oct4 and glial marker GFAP were not detectable. Expression of mesodermal marker SMA was very minimal.

### Example 2

### Harvest and Cryopreservation of Mnps

Materials and Experimental Design

On Day 30 or Day 31, four T-75 flasks were coated with 7 ml per flask of 0.1% gelatin solution and incubated for 30 minutes in the incubator. Approximately 150 ml of MNP induction medium were pre-warmed. The spent medium from the T-75 flasks was aspirated and the flasks were washed once with 15 ml of PBS each. Five ml of TrypLE solution were added and incubated 3-10 minutes. The flask was examined every 3 minutes until most cells were dissociated. Ten ml of MNP induction medium were added to each flask and the suspension was transferred into 50 ml conical tubes. Each T-75 flask was rinsed with an additional 10 ml of medium and the solution was transferred to the 50 ml tubes. The tubes were centrifuged at 200×g for 3 minutes at room temperature. The supernatant was aspirated and the cell pellet was resuspended in 20 ml of medium and the suspension was transferred into the gelatin-coated T-75 flasks (10 ml/flask). The conical tubes were rinsed with 4 ml of MNP induction medium and the solution was transferred to the gelatin-coated T-75 flask. The T-75 flask was incubated for 15 minutes at 37° C. undisturbed. After incubation, all the non-adherent cells were collected from the T-75 flask and transferred to another gelatin-coated T-75 flask, and the flask was gently rinsed with 3-5 ml medium, and the solution was transferred to the gelatin-coated T-75 flasks. The T-75 flasks were incubated for 15 minutes at 37° C. undisturbed. After incubation, all the non-adherent cells were collected from the T-75 flasks and transferred to the 50 ml tubes. The larger cell clumps were allowed to settle to the bottom of the tubes for 3 minutes. The supernatant (>95% of the solution) was transferred into a fresh tube and the cells were counted using a NucleoCounter®. The tubes were centrifuged at 200×g for 3-5 minutes at room temperature. The supernatant was aspirated and 10 ml of cold CryoStor® solution were slowly added to the cell pellet and the cell pellet was gently resuspended. About 100-200 μl of suspension were taken for counting again using NucleoCounter®. The remaining cell solution was put on ice while waiting for cell counts. The viable cell concentration was adjusted to 6 million/ml by adding more CryoStor® solution, and 1 ml suspension was aliquoted into each cryovial. A programmable controlled rate freezer was used to freeze MNPs using the following parameters. After the freezing cycle was completed, cryovials were transferred to liquid nitrogen Dewar immediately after the freezing program was completed

Results

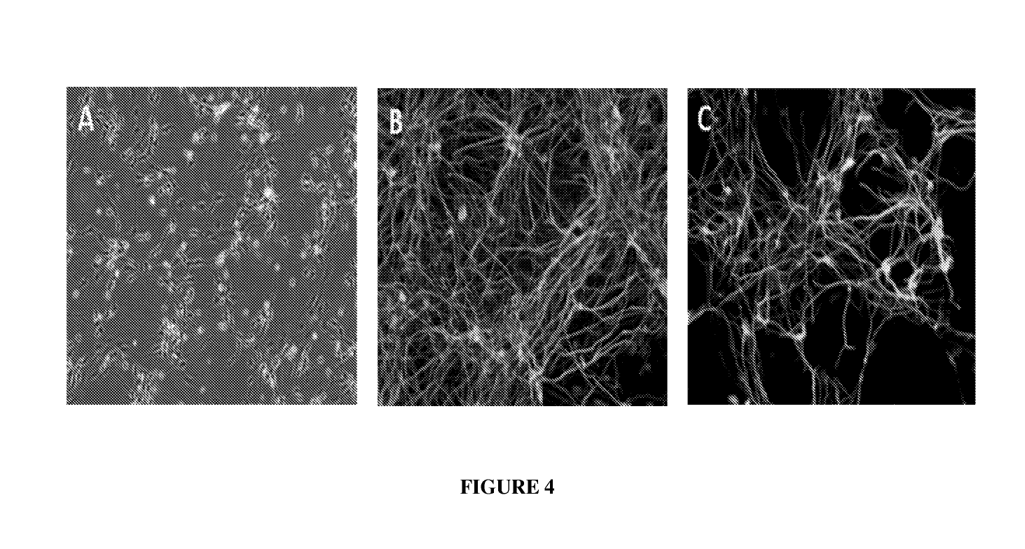

Previous studies of cryopreservation of MNPs by typical method using a freezing container together with a −80° C. mechanical freezer showed inconsistent recovery after thawing and cell viability about 70-80%. The possible explanation is that cryoprotectant was not utilized and cooling rate might not be consistent due to a number of reasons such as location within the −80 C mechanical freezer. To improve recovery of MNP during cryopreservation process, cryoprotectants were examined together with controlled rate freezer to ensure the cooling rate was consistent. Several cryoprotectants such as trehalose, mannitol, and hetastarch were used in combination with DMSO and compared with CryoStor CS10 (Table 1). Cell counts prior to freezing were shown in Table 1 and all tested conditions had cell viability about 90% prior to freezing. Approximately 6×106 cells were frozen in each cryovial. All the cryovials were frozen by using a programmable controlled rate freezer with freezing parameters indicated in Table 2. After storing the cryopreserved MNPs under liquid nitrogen, cells were quickly thawed from liquid nitrogen storage by using a 37° C. water bath. Cell count was determined by Nucleocounter and viability was calculated. The results showed that CryotoR performed better than that of other tested conditions and cell viability was consistently greater than 85 percent after thawing (TABLE 3). Motor neuron markers such as Isl1 and HB9 expression of thawed MNP was normal (FIG. 4B, FIG. 4C) as compared with MNP that did not undergo freezing/thawing (Data not shown).

TABLE 1
Testing freezing media for cryopreservation of MNPs.

| Tested | | Cell Counts | Viabil- |
|---|---|---|---|
| Condi- | | pre- freezing | ity pre- |
| tions | Components | per mL | freezing |
| Control | MNP basal medium + | Viable: 6.18 × 10⁶ | 92% |
| | B27 and 10% DMSO | Total: 6.7 × 10⁶ | |
| KOSR | MNP basal medium + 25% | Viable: 6.19 × 10⁶ | 92.3% |
| | KOSR + B27 and 10% | Total: 6.7 × 10⁶ | |
| | DMSO | | |
| Treha- | MNP basal medium + 5% | Viable: 7.1 × 10⁶ | 93% |
| lose/ | trehalose + 5% mannitol + | Total: 7.65 × 10⁶ | |
| mannitol | B27 and 10% DMSO | | |
| Heta- | MNP basal medium + 6% | Viable: 5.98 × 10⁶ | 93% |
| starch | hetastarch and 10% DMSO | Total: 6.4 × 10⁶ | |
| CryoStor | Proprietary formulation | Viable: 5.72 × 10⁶ | 95% |
| | | Total: 6 × 10⁶ | |

TABLE 2
Freezing parameters for cryopreservation of MNP.

| Conditions | Temperature | Rate |
|---|---|---|
| 1. Holding | 5° C. | |
| 2. Cooling | −5-10° C. | 0.5-2° C./min |
| 3. Ramping | −35-50° C. | 10-30° C./min |
| 4. Ramping | −10-20° C. | 5-20° C./min |
| 5. Cooling | −30-50° C. | 0.5-2° C./min |
| 6. Ramping | −70-90° C. | 5-15° C./min |
| 7. Holding | −70-90° C. | |

### Example 3

### Plating of Motor Neuron

Materials and Experimental Design

Plate Coating

Each 96 well plate required 10 ml of poly-D-lysine working solution (50 μg/ml). The required amount of poly-D-lysine working solution was transferred into a sterile reagent reservoir. Using a multi-channel pipettor, each well of a 96 well plate was coated with 100 μl of poly-D-lysine working solution and the plates were placed in the incubator overnight. After incubation, the poly-D-lysine solution was aspirated using a multi-channel aspirator. The wells were rinsed twice with 100 μl per well of PBS. The plates were dried in the laminar flow hood with the lids off for at least 1 hour and were ready for laminin coating. The required amount of laminin working solution (15 μg/ml) was prepared in MNP basal medium (without B27/NSF1). Using a multi-channel pipettor, 75 μl of laminin working solution were added per well. The plates were incubated at 37° C. for at least one hour but no longer than six hours. The laminin solution was aspirated when the cells were ready to plate. The wells were rinsed with 100 μl of medium per well once prior to plating the cells.

Thawing MNPs

One ampule was thawed in a 37° C. water bath. To insure a good recovery of the cells from cryoperservation, the cell suspensions were thawed quickly but not allowed to sit at or warm above at 37° C. Immediately after the suspension was thawed, the cells were transferred to a 15 ml tube and 9 ml of warmed plating medium (MNP basal medium with NSF1 and glutamine) were added drop by drop in about 2 minutes. The tubes were centrifuged at 200×g for 5 minutes at room temperature. The supernatant was aspirated, the cells were resuspended in 2 ml of fresh plating medium, and 200 μl of cell suspension were counted in the NucleoCounter®. The number of cells required at 40,000 viable cells per well and the cell suspension required for plating were calculated. The calculated cell suspension to have a final concentration of 40,000 viable cells per 300 μl of plating medium was resuspended. The cell suspension was transferred to a sterile reservoir and using a multi-channel pipet 300 μl of the cell suspension were added per well of the MNP. The 96 well plates were placed in the incubator. The plates were left undisturbed for at least 24 hours. After 48 hours of plating, using a multi-channel pipettor, 200 μl of the spent medium were removed from each well and 200 μl of fresh MNP maintenance medium were added. The feeding procedure was done very gently. Extreme care was taken to avoid disturbing the adherent cells. Plates that passed QC testing were fed every other day as described until ready for downstream use.

Results

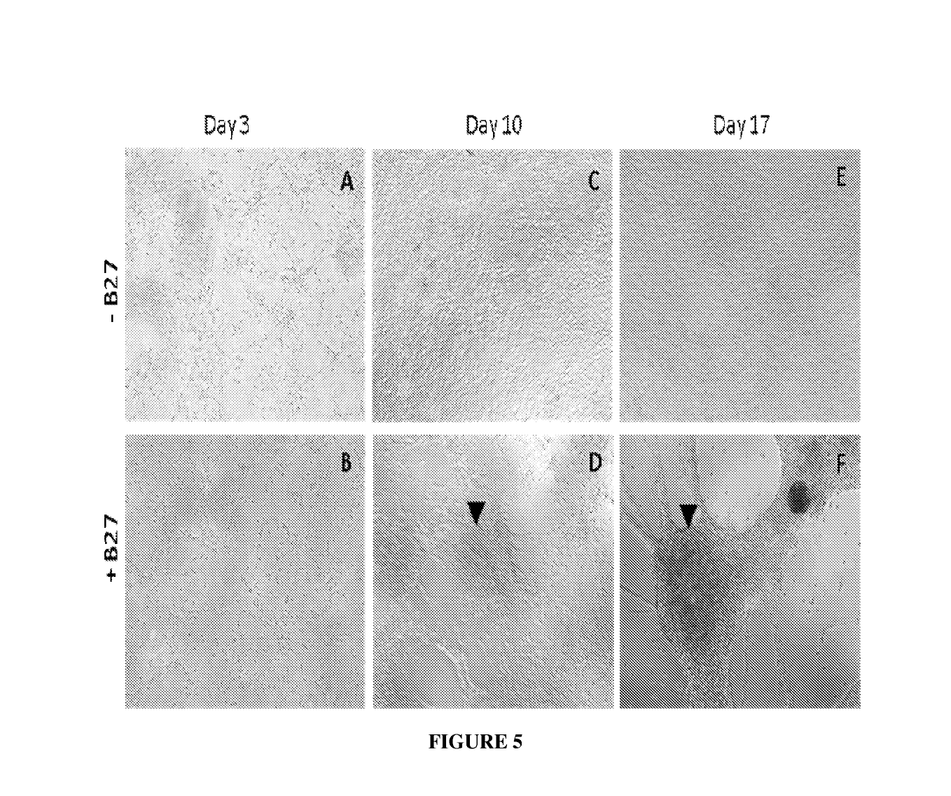

Previous results indicated that MNPs have tendency to clump after about 7-10 days during maturation process. The main reason for this clump problem was unclear. After analyzing the coating procedure of poly-D-lysine and laminin of 96-well plate for MNP plating, laminin coating step used a medium that containing B27. This step created and mixture of B27 and laminin that compete with each other to bind to poly-D-lysine surface and yield a non-homogenous surface for MNP plating. It is well-known that neurons in general are plated on the laminin and poly-D-lysine surface. Our hypothesis is B27 present in laminin coating step causes the MNP clump. A maturation MNP experiment was carried out using either poly-D-lysine coated with laminin alone or laminin+B27 mixture. The cultures were monitored at various time points during the maturation process. The results showed that after 10 days, clumping process started to appear in the culture of Laminin+B27 (FIG. 5D). The clumping became more apparent in MNP culturing on laminin+B27 after 17-21 days (FIG. 5F) while there was no clump detected in culture with laminin coating alone (FIG. 5C and FIG. 5E). These results were consistent with our hypothesis that the B27 is the cause for the MNP clumping during the maturation process.

### Example 4

### Recordkeeping

A batch record was initiated at Day 0 for every non-clinical MNP initiation and was maintained by the production department. Quality records were retained for GMP and ISO requirements as specified in internal standard operating procedures for the retention of quality records. Applicable ISO standards were followed and/or referenced.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, since numerous modifications will readily occur to those skilled in the art, it is not desired to limit the invention to the exact construction and operation illustrated and described, and accordingly, all suitable modifications and equivalents may be resorted to, falling within the scope of the invention. Each reference cited herein is hereby incorporated in its entirety.

We claim:

1. A method of producing high purity motor neuron progenitor cells (MNPs) in vitro, comprising:
culturing undifferentiated human embryonic stem cells (hESCs) or human induced pluripotent stem cells (iPSCs) on feeder cells;
feeding hESCs/iPSCs culture with a mixture of hESC medium and MNP induction 24 hours prior to harvesting;
harvesting hESCs/iPSCs colonies using a passaging solution;
initiating the neuralization step by transferring hESCs/iPSCs colonies into ultra-low attachment flasks containing MNP induction medium supplemented with retinoic acid and FGF-2 to generate neurospheres, for at least 5 days;
ventralizing hESC/iPSCs neurospheres culture by using a MNP induction medium supplemented with FGF-2 for about 10-12 days;
dissociating said neurospheres into smaller spheres and expanding on adherence flasks with MNP induction medium for at least 4 days as neural rosettes;
dissociating said neural rosettes into a cell suspension using trypsin;
purifying and enriching MNPs using a negative adsorption process with gelatin coated surface and replating non-adherence MNPs onto Matrixgel coated flask;
expanding the MNPs using MNP induction medium supplemented with FGF-2 for about 4-5 days;
harvesting adherence MNPs from said matrix coated flasks using trypsin;
purifying the MNPs cell suspension by using negative adsorption process with gelatin-coated flasks; and
collecting resulting non-adherent MNPs from said gelatin-coated flasks.

2. The method of claim 1, wherein the harvested MNPs purity is at least 75-95%

3. The method of claim 1, wherein said feeder cells are mouse embryonic fibroblasts, human dermal fibroblasts or human foreskin fibroblasts.

4. The method of claim 1, wherein the concentration of said bFGF is about 10 ng/ml on the first day of plating of said undifferentiated hESC or iPSC through day 7 of culture, and is about 5 ng/ml on day 8 of culture until harvesting of MNPs.

5. The method of claim 1, wherein the concentration of said retinoic acid is about 10 μM on day 1 of culture through day 7 of culture.

6. The method of claim 1, wherein the mixture of hESC medium and MNP induction medium is about a 1:1 ratio

7. The method of claim 1, said hESC medium comprising Knockout DMEM, 20% KOSR, amino acids, and growth factor

8. The method of claim 1, said MNP induction medium comprising a 50:50 mixture of classical DMEM high glucose and DMEM/F12, Wherein said DMEM/F12 is supplemented with insulin, transferrin, selenium, glutamine, magnesium chloride and B27.

9. The method of cryopreserving MNP comprising the following steps:
(a) centrifuging harvested MNPs-containing supernatant;
(b) resuspending the resulting pellet of MNPs in a freezing medium and aliquoting into a cryovial;
(c) transferring said cryovials containing MNPs to a controlled rate freezer;
(d) subjecting said cryovials containing MNPs to a programmed freezing process in said controlled rate freezer; and
(e) transferring said cryovials containing MNPs from said controlled rate freezer to a liquid nitrogen Dewar following said programmed freezing for long term storage.

10. The method of claim 9, where the recovery cryopreserved MNP viability is about 85-95% after thawing.

11. The method of claim 9, wherein harvested MNPs purity is at least 80-90% prior to cryopreservation.

12. The method of claim 9, wherein the freezing medium is an optimized, cGMP produced, protein-free and serum-free freezing medium pre-formulated with DMSO.

13. The method of claim 9, wherein the freezing medium is CryoStor CS10.

14. The method of claim 9, further comprising the following parameters:
a) equilibrating the said cryovials to 5° C. by using a programmable controlled rate freezer;
b) cooling the said freezer at a rate 0.5-2° C./min until the freezer chamber temperature reaches about 5 to −10° C.;
c) cooling the said freezer at a rate 20-30° C./min until the freezer chamber reaches a temperature of about −35° C. to −50° C.;
d) warming the freezer chamber to 610° C. to −20° C. at a rate of 5° C./min to 20° C./min;
e) cooling the said freezer chamber to −30° C. to 50° C. at a rate of 0.5 to 2° C./min;
f) cooling the said freezer chamber to −70° C. to −100° C. at a constant rate of PC/min to 15′C/min; and
g) removing the cryovials containing MNPs from the said freezer and transferring to liquid nitrogen storage.

15. A method of producing high purity motor neuron progenitor cells (MNPs) in vitro under cGMP comprising:
culturing undifferentiated human embryonic stem cells (hESC) or human induced pluripotent stem cells (iPSC) under feeder free condition for at least 5 days to reach approximately 60-80% confluence;
feeding hESCs/iPSCs culture with a serum free and xeno-free and fully defined medium for 24 hours prior to harvesting;
harvesting hESCs/iPSCs colonies using a protein free non-enzymatic passaging solution;
initiating the neuralization step by transferring hESCs/iPSCs colonies into ultra-low attachment flasks containing MNP induction medium supplemented with retinoic acid and FGF-2 to generate neurospheres for at least 5 days;
ventralizing hESC/iPSCs neurospheres by using a MNP induction medium supplemented with FGF-2 for about 1.0-12 days;
dissociating said neurospheres into smaller spheres and expanding on adherence flasks with MNP induction medium for at least 4 days as neural rosettes;
dissociating said neural rosettes into a cell suspension using trypsin;
purifying and enriching MNPs using negative adsorption process with gelatin coated surface;
replating non-adherence MNPs onto protein free and defined matrix-coated flask;
expanding the MNPs using xeno-free and defined MNP induction medium supplemented with FGF-2 for about 4-5 days;
harvesting adherence MNPs from said protein free and defined matrix-coated flasks using protein free and defined passaging solution;
purifying the MNP cell suspension by using negative adsorption process with gelatin-coated flasks; and
collecting resulting non-adherent MNPs from said gelatin-coated flasks.

16. The method of claim 15, wherein the harvested MNPs purity is at least 60-90%

17. The method of claim 15, wherein the concentration of said bFGF is about 10 ng/ml on the first day of plating of said undifferentiated hESC or iPSC through day 7 of culture, and is about 5 ng/ml on day 8 of culture until harvesting of MNPs.

18. The method of claim 15, wherein FGF-2 is cUMP grade material.

19. The method of claim 15, wherein the concentration of said retinoic acid is about 10 μM on day 1 of culture through day 7 of culture.

20. The method of claim 15, where in the mixture of fully defined and xeno-free medium and MNP induction medium is about a 50:50 ratio.

21. The method of claim 15, wherein fully defined and xeno-free medium is containing vitamins, recombinant albumin, non-essential amino acids, and cGMP grade growth factors.

22. The method of claim 15, wherein MNP induction medium is a 50:50 mixture of classical DMEM high glucose and DMEM/F12, wherein said DMEM/F12 is supplemented with insulin, transferrin, selenium, glutamine, magnesium chloride and B27.

23. The method of claim 22, wherein B27, insulin, FGF-2, and transferrin are cGMP grade materials.

24. The method of claim 1, wherein culturing is for at least 5 days.
